(19) United States
(12) Patent Application Publication
(10) Pub. No.: US 2008/0021475 A1
(43) Pub. Date: Jan. 24, 2008

(54) Orthopaedic appliances

(76) Inventor: Steven Alan Lawrie, Buderim (AU)

Correspondence Address: MICHAEL MOLINS;MOLINS & CO., SUITE 5, LEVEL 6, 139 MACQUARIE ST, SYDNEY NSW, 2000, omitted

(21) Appl. No.: 11/491,116

(22) Filed: Jul. 24, 2006

(51) Int. Cl. A61F 2/30

(52) U.S. Cl. 606/69

(57) ABSTRACT

Provided is a bone plate for the olecranon of the proximal ulna using a combination of screws and K wires. The plate is curved to fit the shape of the olecranon and is screwed in place. The plate has a locking section located in the curved section. A fastener is pushed over the ends of the K wires and locks in to the locking section of the plate. The fastener has a tapered wedge that secures the K wires and resists their withdrawal. A femoral stem cap is used during hip replacement surgery and protects the Morse taper of the femoral stein once the ball of the hip joint is removed. The femoral stem cap has an air vent to allow air to pass when being applied and removed.

### FIELD OF THE INVENTION

The invention relates generally to orthopaedic appliances and more specifically to a plate and Kirshner-wire (K wire) system used for fixation of fractures of the olecranon. Also disclosed is a protective end cap for the femoral stem in hip replacement surgery.

### BACKGROUND AND RELATED ART

Certain types of fractures of the upper arm bones cannot be effectively healed without the use of a bone screw, pin, cable, wire or plate. The use of each of these is known to the prior art.

One of the most common fractures of the elbow involves the olecranon, the proximal end of the ulna. In the most typical fracture pattern, the olecranon is severed from the rest of the ulna along the bottom of the trochlear notch. Such fractures are often difficult to treat because the powerful muscles attached to the olecranon tend to pull it away from the rest of the ulna and rotate it around the trochlea. It is necessary to reattach the severed olecranon to the ulna to immobilize and stabilize the bone until healed.

Another common orthopaedic procedure is a hip replacement with a ball and socket artificial or prosthetic hip joint. It is common that an artificial or prosthetic hip joint needs servicing after several years of use. Sometimes the socket is replaced and not the ball. The ball in some replacement hip joints is held onto a stem by a friction fit called a “Morse taper”. When replacing the socket, the ball is removed from the femoral stem so it is not in the way during surgery. With the ball removed, the Morse taper is vulnerable to damage such as scratching. What is needed is a protective cover or cap for the femoral stem during hip replacement procedures.

### OBJECTS AND SUMMARY OF THE INVENTION

The olecranon plate is a segmented metal plate with holes to accept cooperating screws and K wires. The olecranon is fixed in place with two K wires that are held in place by a threadless friction type fastener that is applied once the plate and K wires are in place. The fastener prevents the K wires from backing out resulting in a loss of fixation.

It is one object of the present invention to provide an olecranon plate for the fixation of fractures of the elbow. It is another object to provide a plate that fixes the olecranon or other bone using K wires, and a threadless fastener that is applied after the K wires are in place, preventing the K wires from backing out of the plate. It is another object to provide a plate that can be used for all fractures and osteotomies of the olecranon.

It is also an object to provide a threadless or friction type fastener that can be used to secure a K wire in a variety of circumstances.

The femoral cap is a disposable plastic cap shaped to provide a sterile protective cover for the femoral end during hip replacement surgery. It is placed over the femoral Morse taper after the ball is removed from the femoral stem. It remains protectively in place while revising the socket. A flange at the cap base allows the cap to be easily removed when time to reinsert the ball once the socket is revised. A vent in the top of the cap allows air to escape when the cap is fitted over the femoral stem. The cap has an internal diameter to fit tapers in common use.

### BRIEF DESCRIPTION OF THE DRAWINGS

FIG. 1 is a side elevation of an olecranon plate and olecranon

FIG. 2 is a perspective view of an olecranon plate with locking section

FIG. 3 is a top plan view of an olecranon plate with locking section

FIG. 4 is a perspective view of the K wires when in place with the locking section and fastener

FIG. 4a is a cross section view of the K wires, locking section and fastener

FIG. 5 is a cross section view of the fastener engaging the K wires

FIG. 6 illustrates an inverted perspective view of the fastener

FIG. 7 is a perspective view of the olecranon plate

FIG. 8 illustrates a cross sectional view of the femoral stem cap

FIG. 9 is a top plan view of a further embodiment of an olecranon plate

FIG. 10 is a top plan view of a threaded fastener

FIG. 11 is a perspective view of the fastener depicted in FIG. 10

FIG. 12 is a detailed plan view of the olecranon plate, K wires and fastener

### DETAILED DESCRIPTION OF THE PREFERRED EMBODIMENTS

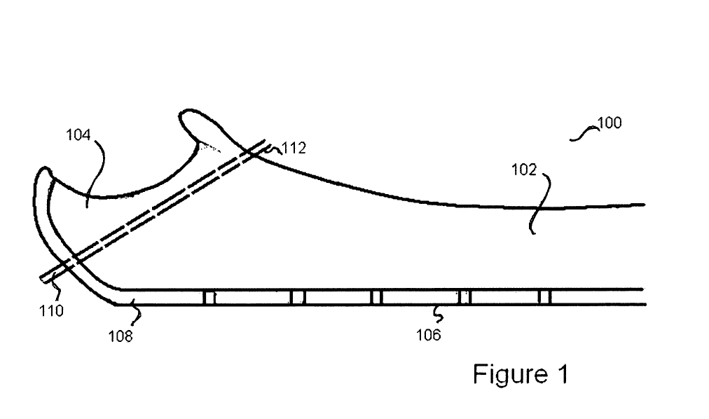

The bone plate of the present invention is adapted for fractures of the olecranon but the principles taught herein, particularly with regard to the fastening of K wires, may have application in a variety of K wire fixation systems. FIG. 1 illustrates the ulna bone 102 and olecranon 104. The olecranon plate 106 is attached to the ulnar shaft using screws (not shown). One end of the olecranon plate is bent to shape the curve of the ulna 108. One or more, and in this example two laterally spaced, parallel K wires 112 pass through the olecranon plate 110 and into the ulna.

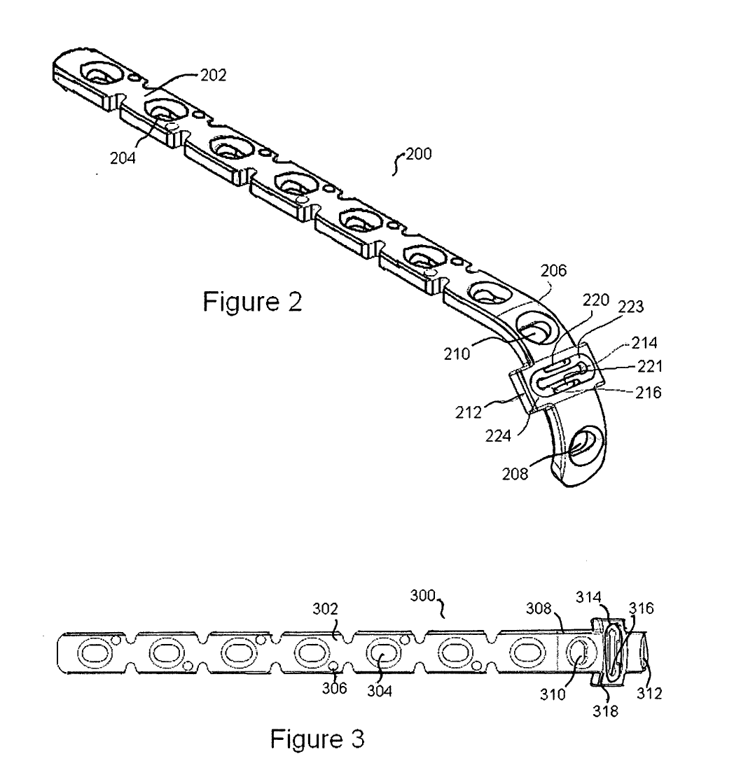

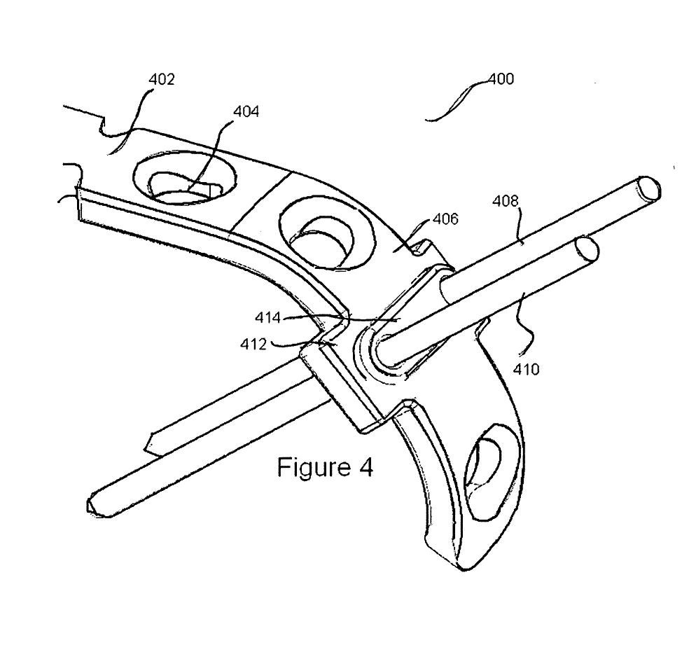

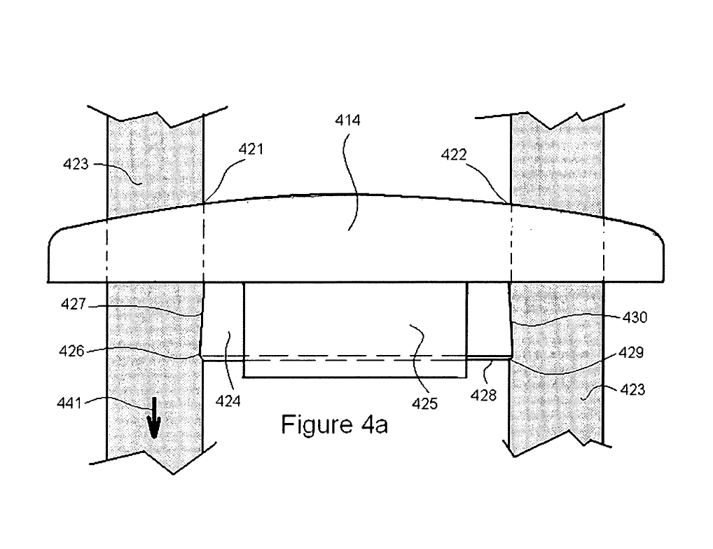

As shown in FIGS. 2 and 3, the plate comprises a notched or segmented shaft portion 202, with at one or more counter-bored shaft screw hole 204 for cooperating fixation screws. Opposing notches that define the segments allow the shaft to be bent, as required. The screw or screws are used to attach the plate to the shaft of the ulna. The plate 202 has a curved end 206 with two screw holes 208 and 210 that are used hold the curved portion against the curved section of the ulna. A rectangular locking section 212 lies, for example, between these last two screw holes and has a slot formed by two interconnected openings 214 and 216. In this example the locking section is wider than the remainder of the plate, providing optimum spacing for the K wires. The openings are for receiving the K wires. The slot also receives the wedge 424 of the fastener (see FIG. 4). The slot 214, 216 is flanked by a pair of generally parallel and rectangular openings 220, 221. The openings 220, 221 and the slot 214, 216 are contained within the sidewalls of shallow well 223 having rounded ends 224.

FIG. 4 illustrates the locking section 412 with K wires 408 and 410 and fastener 414 in place. The fastener is threadless and acts like a clamp, resisting the movement of the K wires through the fastener in one direction more than the other. The fastener 414 slides into position within the well in the locking section by pushing it over the ends of the K wires once they are in place.

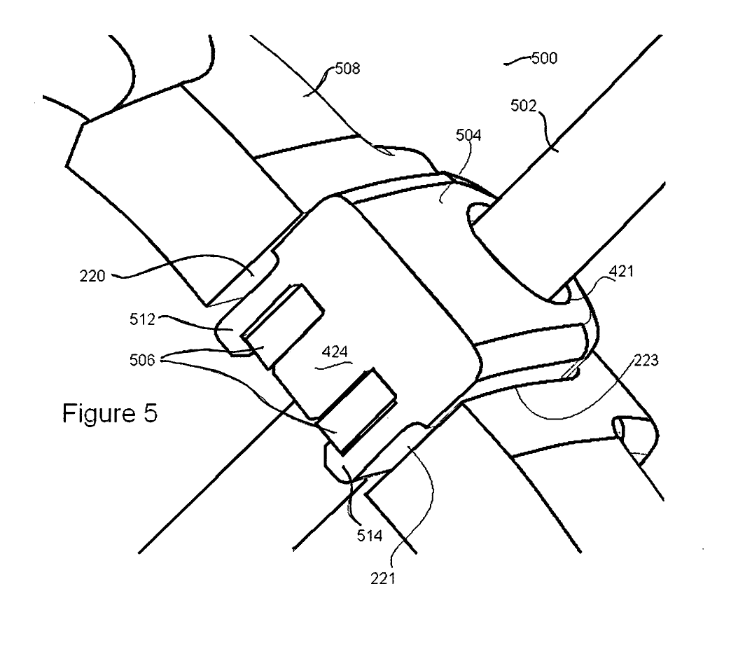

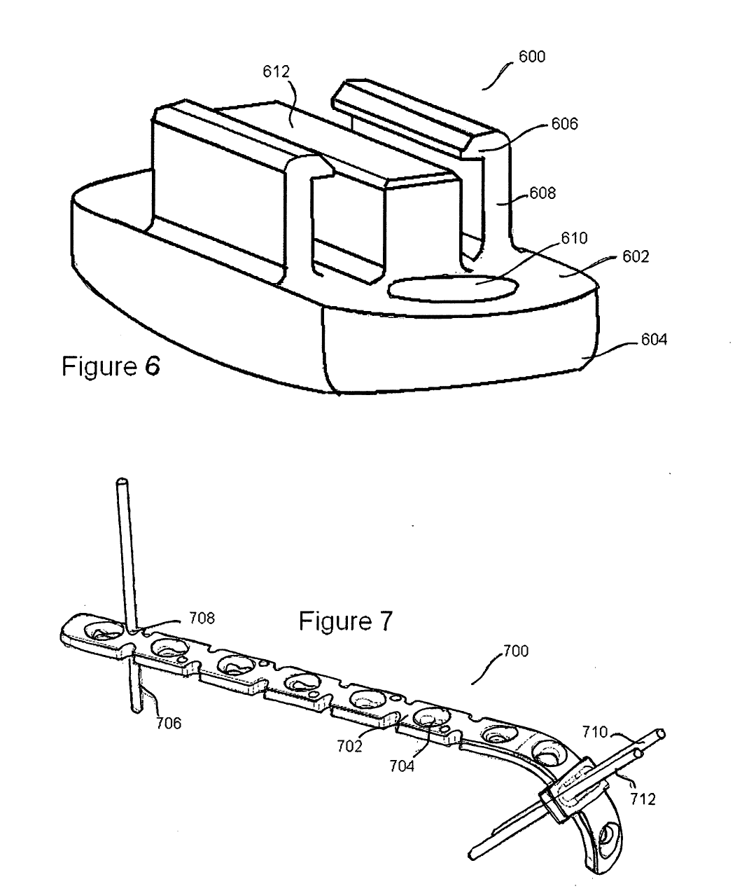

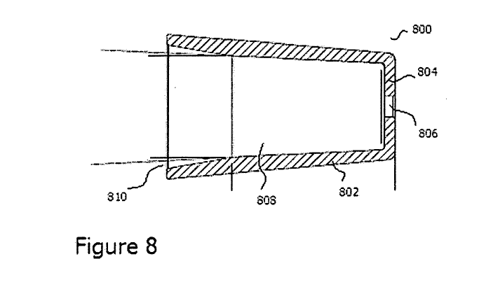

As shown in FIG. 4a (and with reference to FIGS. 5 and 6), the clamping fastener 414 comprises a cap 604 with smooth upper edges that is received within the well of the locking section. The cap has two through holes 421, 422, 610 in it, each one receiving a K wire 423. A lower surface of the cap has extending from it a rigid locking wedge 424 and a pair of locking tabs 425 that deflect, slide into, and engage the openings 420, 421. The locking wedge and tabs are located between the holes 421, 422. The holes 421, 422 are in registry with the slanted lateral sides of the wedge such that the slanted sides 427 make contact with the K wires. The slanted edges have a protruding gripping edge 426 located toward the bottom extent 428 of the wedge. As shown in FIG. 4a, the short portion 429 of the wedge's side, below the edge 426, forms a sharper angle with respect to the K wire than the portion of the side 430 above the edge 426. In this way, the K wire is more easily drawn in one direction 441 than in the opposite direction. This feature resists inadvertent withdrawal of the K wires from the fastener 414.

As shown in FIGS. 5 and 6, the clamping fastener 504 is shown as having two parallel tabs 512 with inwardly directed flanged tips 514 that enter the openings 220, 221, by deflecting, then snap over cooperating locking bars 506 in the locking section. The locking bars are located between the slot 214, 216 and the two openings 220, 221. This action secures the fastener to the plate.

The plate of the present invention allows for the use of either screws or K wires for proximal fixation. FIG. 7 illustrates a lateral view of the olecranon plate with a K wire 706 inserted through a receiving hole thus fixing the plate to the ulnar shaft.

The olecranon plate of the present invention is anatomically specific for the fixation of olecranon fractures of the elbow. It is metal, for example, 316L stainless steel. It may use conventional dimensions for DCP screw holes, and provisional fixation holes.

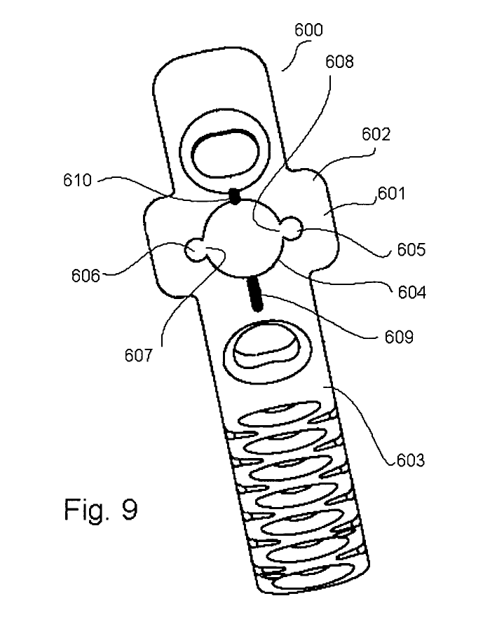

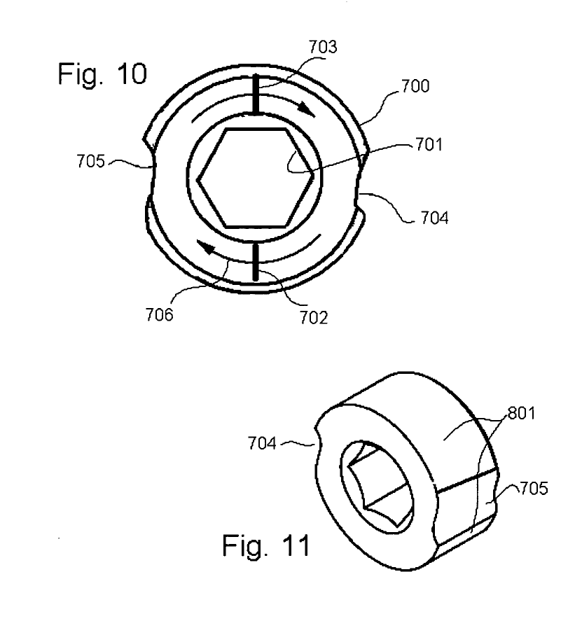

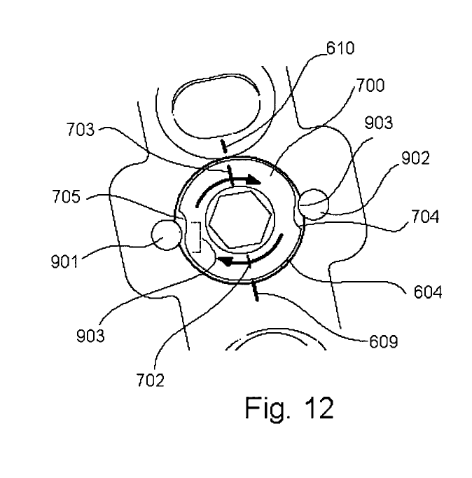

Another embodiment of the olecranon plate of the present invention is depicted in FIGS. 9-12. As shown in FIG. 9, the olecranon plate 600 includes an enlarged locking section 601 that includes lateral ears 602 that project from the side edges of the main body 603. A threaded opening 604 is formed in the central part of the locking section 601. The treaded opening 604 is adapted to receive a fastener of the type that will be disclosed with reference to FIGS. 10 and 11. Additional through holes 605, 606 are provided for the K wires. The K wire holes 605, 606 intersect the side walls of the threaded opening 604 thereby defining a gap 607, 608 that allows the exterior surfaces of the K wires to actually protrude through the threaded side walls of the threaded opening 604. This is shown in FIG. 12. Note that the top surface of the body 603 is provided with diametrically opposed, preferably laser etched, orientation marks 609, 610.

A fastener that is particularly adapted to the olecranon plate depicted in FIG. 9 is shown in FIGS. 10 and 11. The fastener is for example based on an M8×1 hex head grub screw type fastener, particular one that is adapted to fit a Synthesis 3.5 mm hex driver. The fastener 700 is thus provided with a hexagonal shaped driver hole 701 and diametrally opposed orientation marks 702, 703 that align with the orientation marks 609, 610 on the olecranon plate 600. When the alignment marks (e.g. 702, 703, 609, 610) are in alignment, longitudinal grooves 704, 705 will be in registry with the K wire gaps 607, 608. This particular orientation is depicted in FIG. 12. Like the alignment mark 702, 703, the upper surface of the fastener 700 is provided with laser etched arrows 706 that indicate clockwise rotation of the fastener which is the correct rotation for tightening the fastener against the K wires. It will be appreciated that the fastener 700 is provided with external threads although these threads are either not formed or obliterated in the area of the grooves 704, 705. As shown in FIG. 11, the shaded area 801 indicates the threaded external portion of the fastener 700. The groove areas 704, 705 need to be deep enough that the protruding portions the K wires are received by the grooves 704, 705 as shown in FIG. 12.

As shown in FIG. 12, the fastener is used by first inserting and threading it into the threaded opening 604 when it is sufficiently threaded in to be within about a quarter turn of its final orientation and with the fastener's alignment marks 702, 703 in alignment with the body's alignment marks 609, 610. The resulting sub-assembly is ready for receiving the K wires 901, 902. Note that a portion 903 of the proximal end of the K wire protrudes through the gap 608 and into the area of the groove 704. In this orientation, the fastener does not interfere or necessarily even contact the K wire. Upon rotation of the fastener, by about say one quarter of a turn, the sharp threaded exterior surface of the fastener gradually begins to make contact with the K wire by actually cutting into the body of the K wire creating a mechanical interlocking of the fastener with the K wires. In preferred embodiments there is a transition zone 905 where the radius of the exterior of the fastener increases gradually from its minimum at the base of the groove 704, 705 out to the maximum radius of the fastener. This allows the threads to be gradually introduced into the K wire and thus facilitating locking of the fastener into the metallic bodies of the K wires.

Femoral Stem Cap

The femoral stem cap is a plastic protective cap that is placed over the femoral stem after the ball has been removed during total hip replacement surgery. The stem cap is round in cross section and internally tapered to fit snugly over, for example, the 12-14 Euro taper or Morse taper of the stem trunion. It may be externally tapered to reduce material consumption during manufacture. FIG. 1 illustrates a cutaway lateral view of the stem cap. The cap 800 has a sidewall 802 and a top wall 804. The top wall has a central round vent 806 that allows the escape of air while positioning the cap over the femoral stem. The interior of the cap 808 features a Morse taper that matches the taper on the femoral stem. The cap edge has a piloted opening 801 that allows for easy insertion and removal. The femoral stem cap is sized to fit the common sizes of trunion tapers used. It is made of plastic and is preferably sterile when applied.

We claim:

1. An olecranon plate, comprising:
a bone plate, having a curved portion adapted to the curvature of the olecranon, the curved portion having formed in it a locking section for receiving a clamping fastener, the locking section and fastener adapted to receive one or more K wires.

2. The olecranon plate of claim 1, wherein:
the locking section comprises a fastener opening flanked by a pair of K wire openings, the K wire openings adapted to receive the one or more K wires;
the fastener comprising a threaded body having an at least partially threaded exterior into which exterior is formed one or more longitdinal grooves.

3. The olecranon plate of claim 2, wherein:
the one or more grooves are adapted to receive a portion of a K wire that protrudes between a gap located between the fastener opening and K wire opening.

4. The olecranon plate of claim 1, wherein:
the groove has a tapered section that leads to the threaded exterior of the fastener.

5. The olecranon plate of claim 4, wherein:
the fastener has a hexagonal central opening.

6. The olecranon plate of claim 1, wherein:
a pair of diametrally opposed K wire openings are used with the fastener opening.

7. The olecranon plate of claim 1, wherein:
the locking portion has lateral ears.

8. The olecranon plate of claim 1, wherein:
the locking section is wider than the curved portion.

9. The olecranon plate of claim 1, wherein:
the K wires are two in number and spaced laterally from one another.

10. The olecranon plate of claim 9, wherein:
the one or more grooves are not threaded.

11. A protective stem cap, comprising:
a plastic protective cap, generally round in cross section and internally tapered to fit snugly over a Morse taper.

12. The cap of claim 11, wherein:
the cap is externally tapered.

13. The cap of claim 11, wherein:
the cap has a sidewall and a top wall;

12. The cap of claim 13, wherein:
the top wall has a central vent that allows the escape of air while positioning the cap over a femoral stem.

13. The cap of claim 11, wherein:
the interior of the cap features a Morse taper that matches a taper on a femoral stem.

14. The cap of claim 11, wherein:
the cap has a free edge defining a piloted opening that allows for easy insertion and removal.

15. The cap of claim 11, wherein:
the cap is sterile.
